(19) United States
(12) Patent Application Publication
(10) Pub. No.: US 2009/0068279 A1
(43) Pub. Date: Mar. 12, 2009

(54) MICROSPHERES WITH SURFACE PROJECTIONS

(75) Inventor: Robert E. Richard, Wrentham, MA (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/187,761

(22) Filed: Aug. 7, 2008

Related U.S. Application Data
(60) Provisional application No. 60/971,788, filed on Sep. 12, 2007.

(51) Int. Cl. A61K 9/14; B32B 15/02

(52) U.S. Cl. 424/497; 428/407

(57) ABSTRACT

Methods of making polymer particles, as well as related particles, compositions, and methods are disclosed.

### CROSS-REFERENCE TO RELATED APPLICATION

This application claims priority under 35 U.S.C. §119 to U.S. Ser. No. 60/971,788, filed Sep. 12, 2007, the contents of which are hereby incorporated by reference.

### TECHNICAL FIELD

This disclosure relates to methods of making polymer articles, as well as related particles, compositions, and methods.

### BACKGROUND

Agents, such as therapeutic agents, can be delivered systemically, for example, by injection through the vascular system or oral ingestion, or they can be applied directly to a site where treatment is desired. In some cases, particles are used to deliver a therapeutic agent to a target site. Additionally or alternatively, particles may be used to perform embolization procedures and/or to perform radiotherapy procedures.

### SUMMARY

An article includes: a particle including a first polymer, the particle having an outer surface, and the particle having a maximum dimension from 50 to 5,000 microns. A first polymer chain including a second polymer extending radially outward from the outer surface of the particle.

A composition includes: a carrier fluid; and a plurality of particles in the carrier fluid. At least some of the plurality of articles include: a particle including a first polymer, the particle having an outer surface, and the particle having a maximum dimension from 50 to 5,000 microns. A first polymer chain including a second polymer extends radially outward from the outer surface of the particle.

An article includes: a particle including a polymer, the particle having an outer surface, and the particle having a maximum dimension from 50 to 5,000 microns. A fiber member extends radially outward from the outer surface of the particle. In some embodiments, the fiber member is disposed partially within the particle. In some embodiments, the fiber member includes a material selected from the group consisting of cotton, polyethylene terephthalate, nylon, and collagen.

A composition includes: a carrier fluid; and a plurality of articles in the carrier fluid. At least some of the plurality of articles include: a particle including a polymer, the particle having an outer surface, and the particle having a maximum dimension from 50 to 5,000 microns. A fiber member extends radially outward from the outer surface of the particle.

Embodiments of the articles and compositions can include one or more of the following features.

In some embodiments, the second polymer is soluble in water at 20 degrees Celsius. In some embodiments, the second polymer is insoluble in water at 20 degrees Celsius.

In some embodiments, the second polymer has a volume phase transition temperature from 30 to 40 degrees Celsius. In some cases, the second polymer has a volume phase transition temperature from 35 to 40 degrees Celsius.

In some embodiments, the article also includes a second polymer chain including a third polymer. In some cases, the second polymer is soluble in water at 20 degrees Celsius and the third polymer is insoluble in water at 20 degrees Celsius.

In some embodiments, the first polymer includes vinyl alcohol monomers (e.g., at least five weight percent vinyl alcohol monomers).

In some embodiments, the first polymer further includes vinyl acetate monomer units (e.g., at least five weight percent vinyl alcohol monomers).

In some embodiments, the article also includes a therapeutic agent. In some cases, the therapeutic agent is at least partially disposed within pores included in the particle. In some cases, the therapeutic agent is coated on a surface of the particle. In some cases, the therapeutic agent is at least partially disposed within pores in the polymer chain. In some cases, the therapeutic agent is at least partially disposed within pores on the fiber member.

In some embodiments, the first polymer is cross-linked.

In some embodiments, the first polymer is different than the second polymer.

A method includes: forming a particle including a first polymer, the particle having an outer surface, and the particle having a maximum dimension from 50 to 5,000 microns; and forming a polymer chain including a second polymer extending radially outward from the outer surface of the particle. Embodiments can include one or more of the following features.

In some embodiments, the particle includes polymerization initiators exposed on the outer surface of the particle before the polymer chain is formed.

In some embodiments, forming the polymer chain includes graph polymerization of the polymer chain from an iniferter exposed on the outer surface of the particle.

In some embodiments, the method also includes cross-linking the first polymer.

A particle includes: a core having an outer surface, and a core size from 50 to 5,000 microns; and a shell formed on the outer surface of the core. A first area of the shell includes a first polymer and a second area of the shell includes a second polymer different than the first polymer. The first area extends radially outward an average first distance from a center of the core and the second area extending radially outward an average second distance from the center of the core, the average first distance being greater than the average second distance. Embodiments of the articles can include one or more of the following features.

In some embodiments, the particle includes pores extending through the shell. In some cases, the pores extend into the core.

In some embodiments, the first distance is at least 10 percent greater than the second distance. In some cases, the first distance is less than 50 percent greater than the second distance.

A method includes: preparing an aqueous solution containing a first amphiphilic diblock copolymer and a second amphiphilic diblock copolymer, the first amphiphilic diblock copolymer and the second amphiphilic diblock copolymer sharing the same water-insoluble block but having different water-soluble blocks; mixing the aqueous solution with an oil phase to form drops of the oil phase surrounded by a layer including the first amphiphilic diblock copolymer and the second amphiphilic diblock copolymer; and solidifying the drops and surrounding layers to form particles. The aqueous solution is mixed with the oil phase at rate such that the drops of the oil phase are from 50 to 5,000 microns in size and the aqueous solution is mixed with the oil phase until the first amphiphilic diblock copolymer substantially migrates to first areas of the layer and the second amphiphilic diblock copolymer substantially migrates to second areas of the layer. Embodiments of the articles can include one or more of the following features.

In some embodiments, solidifying the drops and surrounding layers includes heating the drops and surrounding layers.

In some embodiments, the method also includes cross-linking the particles

A method of treating an individual includes: placing a therapeutically effective amount of particles including a first polymer in a tissue of the individual, the particles having outer surfaces, and the particles having a maximum dimension from 50 to 5,000 microns. First polymer chains including a second polymer extend radially outward from the outer surface of the particles.

A method of treating an individual includes: placing a therapeutically effective amount of particles including a first polymer in a tissue of the individual, the particles having outer surfaces, and the particles having a maximum dimension from 50 to 5,000 microns. Fiber members extend radially outward from the outer surfaces of the particles. In some embodiments, the fiber members are disposed partially within the particles. In some embodiments, the fiber members include a material selected from the group consisting of cotton, polyethylene terephthalate, nylon, and collagen.

A method of treating an individual includes: placing a therapeutically effective amount of particles including a first polymer in a tissue of the individual. Each particle includes: a core having an outer surface, and a core size from 50 to 5,000 microns; and a shell formed on the outer surface of the core. A first area of the shell includes a first polymer and a second area of the shell includes a second polymer different than the first polymer. The first area extends radially outward an average first distance from a center of the core and the second area extends radially outward an average second distance from the center of the core, the average first distance being greater than the average second distance.

Embodiments of the methods of treating can include one or more of the following features.

In some embodiments, the particles are placed percutaneously.

In some embodiments, the particles are placed through a catheter.

Embodiments can include one or more of the following advantages.

Embolic articles can be formed with surface features to impart enhanced thrombogenicity, aggregation properties, swelling, drug delivery capabilities and potential for use with fibered coil devices. For example, articles can include polymer chains extending outward from the surface of particles with the size, number, and chemical nature (e.g., ionic nature) of the polymer chains chosen to modulate the release of a specific therapeutic agent. Similarly, fibers extending out of polymer particles can provide sites promoting thrombosis when a composition containing the particles is used in embolization procedures.

The particles can optionally be used to deliver therapeutic agents within a body lumen, alone or in combination with an embolization procedure.

The methods can provide particles appropriate for use in, for example, embolization and/or therapeutic agent delivery within a body lumen (e.g., a blood vessel of a human or an animal).

The methods can provide particles having certain desirable physical properties for delivery in a body lumen (e.g., a blood vessel), such as, for example, hardness.

Features and advantages are in the description, drawings, and claims.

### DESCRIPTION OF DRAWINGS

FIGS. 1A and 1B are, respectively, a side view and a partially cutaway side view of an embodiment of an article.

FIG. 2 is an illustration of an embodiment of a system and method for producing articles.

FIG. 3 is an illustration of a droplet generator system.

FIGS. 4A and 4B are, respectively, a side view and a partially cutaway side view of an embodiment of a particle.

FIG. 5 is an illustration of an embodiment of a system and method for producing particles.

FIG. 6 shows partially cutaway side views of an embodiment of a particle at different stages of being produced.

FIG. 7A is a schematic illustrating an embodiment of a method of injecting a composition including articles into a vessel.

FIG. 7B is a greatly enlarged view of region 7B in FIG. 7A.

FIGS. 8A and 8B are, respectively, a side view and a partially cutaway side view of an embodiment of an article.

FIG. 9 is an illustration of an embodiment of a system and method for producing articles.

Like reference symbols in the various drawings indicate like elements.

### DETAILED DESCRIPTION

Particles can be used to deliver a therapeutic agent to a target site. Additionally or alternatively, particles may be used to perform embolization procedures and/or to perform radiotherapy procedures. Particles can be modified with surface features (e.g., polymer chains, fibers, or bumps extending from a particle's outer surface) to impart enhanced thrombogenicity, aggregation properties, swelling, drug delivery capabilities, and/or potential for use with fibered coil devices.

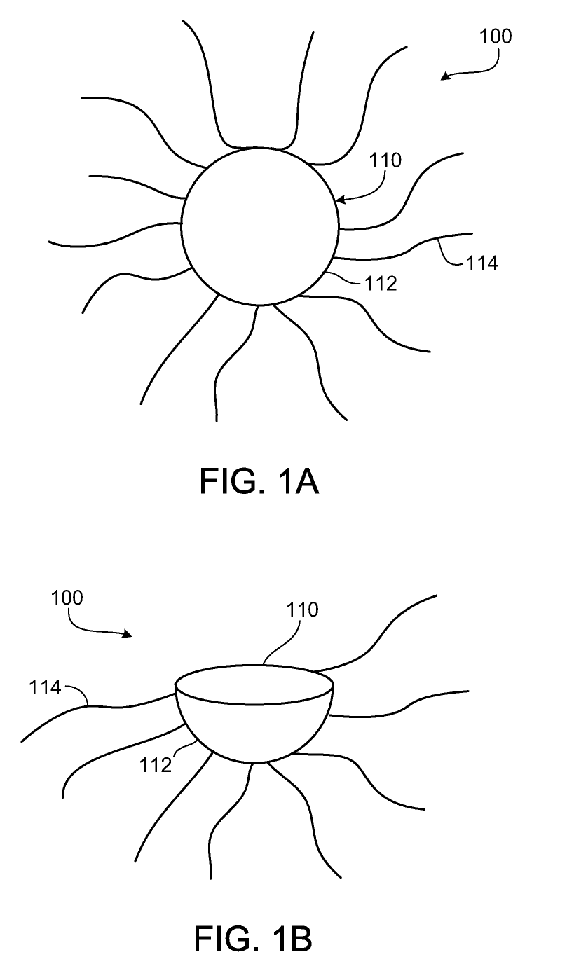

FIGS. 1A and 1B show an article 100 that can be used, for example, in an embolization procedure. Article 100 includes a particle 110 having an outer surface 112 and polymer chains 114 extending outward from outer surface 112 of particle 110. Particle 110 is formed of a polymer, such as polyvinyl alcohol (PVA). Polymer chains 114 extend outward from iniferters exposed on outer surface 112 of particle 110.

Iniferters are initiators that induce radical polymerization that proceeds via initiation, propagation, primary radical termination, and transfer to initiator. Because bimolecular termination and other transfer reactions are negligible, these polymerizations are performed by the insertion of the monomer molecules into the iniferter bond, leading to polymers with two iniferter fragments at the chain ends. If the end groups of the polymers obtained by a suitable iniferter serve further as a polymeric iniferter, these polymerizations proceed by a living radical polymerization mechanism.

The polymers used can be selected to provide specific characteristics to articles 100. For example, in some embodiments, the second polymer used to form polymer chains 114 has hydrophilic properties. In other embodiments, the second polymer used to form polymer chains 114 has hydrophobic properties. A hydrophilic molecule or portion of a molecule is one that is typically charge-polarized and capable of hydrogen bonding, enabling it to dissolve more readily in water than in oil or other hydrophobic solvents (e.g., water soluble at 20 degrees Celsius). In contrast, hydrophobic/polymers are repelled from a mass of water (e.g., insoluble in water at 20 degrees Celsius). Hydrophobic molecules/polymers tend to be nonpolar and thus prefer other neutral molecules and nonpolar solvents. Hydrophobic molecules/polymers in water often cluster together. Polymers with hydrophilic properties include, for example, polyhydroxyethyl (meth) acrylate, polyvinyl alcohol, polyethylene oxide macromers, poly(meth)acrylamides, polyvinyl pyrrolidone, poly(meth)acrylic acid. Polymers with hydrophobic properties include, for example, polystyrene, polymethyl(meth)acrylate, other polyalkyl(meth)acrylates. In some applications, polymers with a specific hydrophobic/hydrophilic characteristics can be used to selectively dispense therapeutic agents and/or control aggregation characteristics.

The polymers used and made can have a volume phase transition temperature near body temperatures (e.g., from 30 to 40 degrees Celsius, from 35 to 40 degrees Celsius). The volume phase transition temperature of a material is a temperature at which the material substance significantly shrinks or swells (e.g., changes volume by more than 10%, more than 20%, or more than 50%) in response to a small temperature change (e.g., less than 5 degrees Celsius, less than 2 degrees Celsius, or less than 1 degree Celsius). The volume phase transition temperature can be measured by measuring the volume of the material at different temperatures. In some applications, polymers with a volume phase transition temperature near body temperatures can be used to selectively dispense therapeutic agents.

In some embodiments, first and second polymers can be the same polymer. In some embodiments, first and second polymers can be different polymers.

In some embodiments, articles 100 include polymer chains 114 formed of multiple polymers (e.g. at least two polymers, at least three polymers, or at least five polymers). The multiple polymers can be combined in individual polymer chains 114 and/or individual polymer chains 114 can be formed of specific polymers (e.g., some polymer chains 114 can be formed of a second polymer and other polymer chains 114 can be formed of a third polymer). The multiple polymers can have different properties than each other. For example, the second polymer can have hydrophilic properties and the third polymer can have hydrophobic properties.

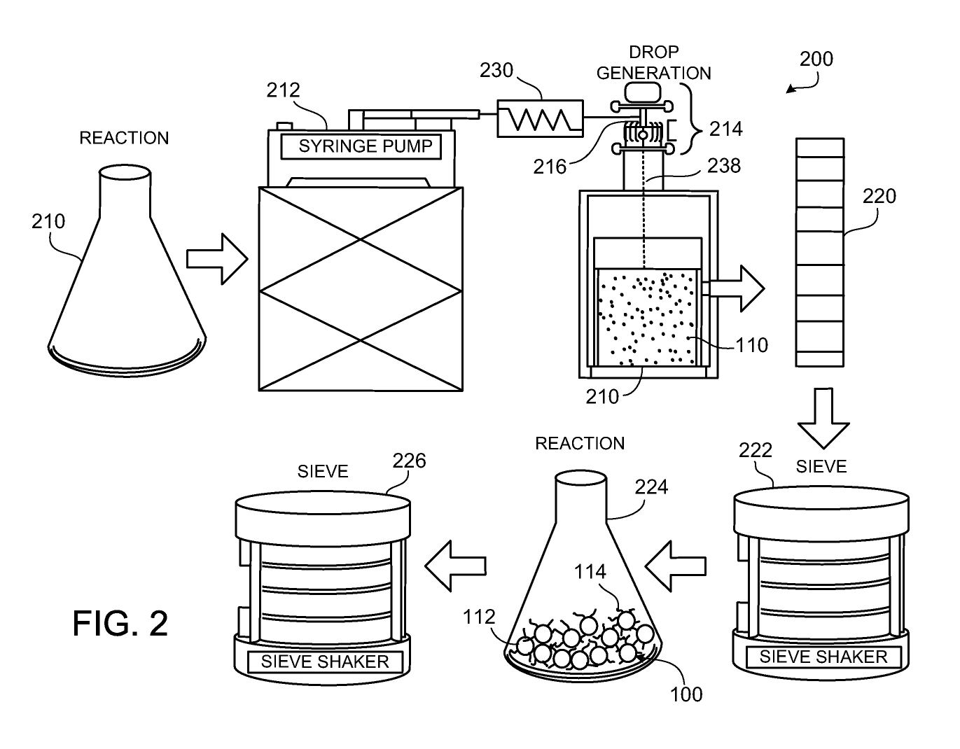

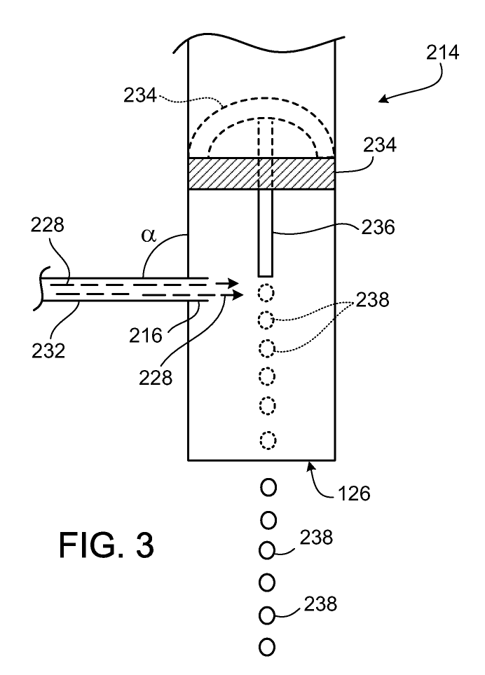

FIGS. 2 and 3 show a system 200 that can be used to produce articles 100. System 200 includes a reaction vessel 210, a flow controller 212, a drop generator 214 including a nozzle 216, a gelling vessel 218, a gel dissolution chamber 220, a filter 222, a second reaction vessel 224, and a second filter 226. An example of a commercially available drop generator is the model NISCO Encapsulation unit VAR D (NISCO Engineering, Zurich, Switzerland). Drop generators are described, for example, in Lanphere et al., U.S. Patent Application Publication No. US 2004/0096662 A1, published on May 20, 2004, and entitled “Embolization”, and in DiCarlo et al., U.S. patent application Ser. No. 11/111,511, filed on Apr. 21, 2005, and entitled “Particles”, both of which are incorporated herein by reference.

A polymer solution including a gelling precursor is mixed with a iniferter (i.e., a chemical that acts as an initiator, a transfer agent, and a terminator in free radical reactions) in reaction vessel 210. The resulting mixture is transferred to the input of flow controller 212. Flow controller 212 includes a high pressure pumping apparatus, such as a syringe pump (e.g., model PHD4400, Harvard Apparatus, Holliston, Mass.). Flow controller 212 delivers a stream 228 of the polymer mixture to a viscosity controller 230, which heats the solution to reduce its viscosity prior to delivery to drop generator 214. In some embodiments, a therapeutic agent can be added before the solution is delivered to viscosity controller 230. Viscosity controller 230 is connected to nozzle 216 of drop generator 214 via tubing 232. After stream 228 has traveled from flow controller 230 through tubing 232, stream 228 flows around a corner having an angle α, and enters nozzle 216. As shown, angle α is about 90 degrees. However, in some embodiments, angle α can be less than 90 degrees (e.g., less than about 70 degrees, less than about 50 degrees, less than about 30 degrees).

As stream 228 enters nozzle 216, a membrane 234 in nozzle 216 is subjected to a periodic disturbance (a vibration). The vibration causes membrane 234 to pulse upward (to the position shown in phantom in FIG. 3) and then return back to its original position. Membrane 234 is connected to a rod 236 that transmits the vibration of membrane 234, thereby periodically disrupting the flow of stream 228 as stream 228 enters nozzle 216. This periodic disruption of stream 228 causes stream 228 to form drops 238. Drops 238 fall into gelling vessel 218, where drops 238 are stabilized by gel formation. During gel formation, the gelling precursor in drops 238 is converted from a solution to a gel form by a gelling agent contained in gelling vessel 218.

Particles 110 formed by gel-stabilized drops 238 are then transferred from gelling vessel 218 to gel dissolution chamber 220. In gel dissolution chamber 220, the gelling precursor (which was converted to a gel) in particles 110 is dissolved. After the particle formation process has been completed, particles 110 can be filtered in filter 222 to remove debris. Filtered particles 110 are transferred to reaction vessel 224 and mixed with an aqueous solution containing monomer units capable of reacting with iniferters exposed on outer surfaces 112 of filtered particles 110 to form polymer chains 114 extending outward from outer surfaces 112. Articles 100 including particles 110 and polymer chains 114 extending from outer surfaces 112 of particles 110 can then be filtered in second filter 226 to remove debris. Optionally, articles 100 can then be cross-linked (e.g., by irradiation with ultraviolet light).

Appropriate iniferters for iniferter-mediated graft radical polymerization include, for example, (Methacryloyl ethylenedioxycarbonyl)benzyl N,N-diethyldithiocarbamate or N,N-diethyldithiocarbamide acetic acid. Other polymerization techniques including “grafting from” techniques using, for example, nitroxide-mediated graft radical polymerization, atom transfer radical polymerization, and reversible additional fragmentation transfer polymerization can also be used to form the polymer chains.

Articles 100 illustrate particles with polymer chains 114 as surface features. Particles can also be formed with other surface features including, for example, bumps extending from a particle's outer surface.

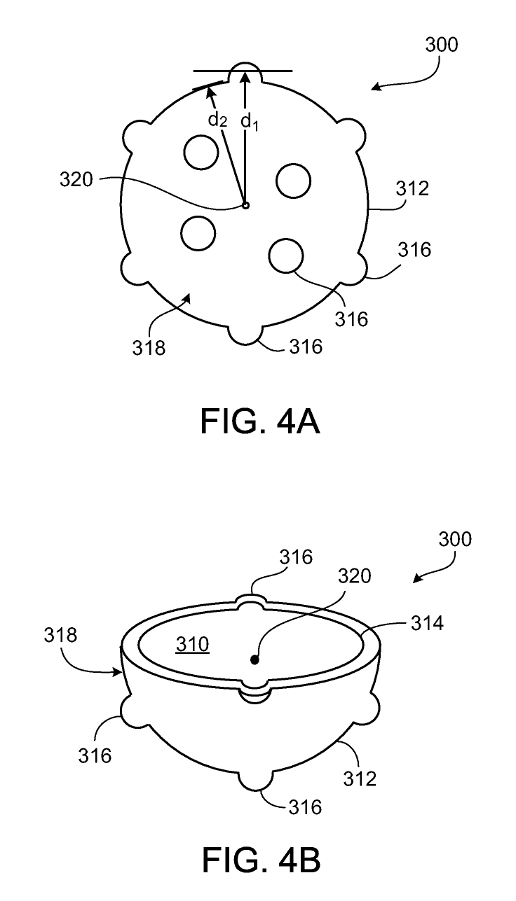

FIGS. 4A and 4B show an embodiment of a particle 300. Particle 300 includes a core 310 and a shell 312. Shell 312 is formed on an outer surface 314 of core 310. Shell 312 has hemispheric first areas 316 including a first polymer and second areas 318 including a second polymer. Each first area 316 extends an average first radial distance d₁ from a center 320 of core 310 that is greater than an second average radial distance d₂ that adjacent second area(s) 318 extends from center 320 of core 310. For example, second radial distance d₂ can be greater than 25 microns (e.g., greater than 100 microns, greater than 500 microns, greater than 1000 microns, greater than 2,000 microns) and/or less than 2,500 microns (e.g., less than 2,000 microns, less than 1,000 microns, less than 500 microns) and/or from 25 to 2,500 microns in size. First radial distance d₁ can be at least at least 10 percent greater than second radial distance d₂ (e.g., 15 percent greater, 25 percent greater, or 50 percent greater) and at most 100 percent greater than second radial distance d₂ (e.g., 75 percent greater, 50 percent greater, or 25 percent greater). In some embodiments, particle 300 includes pores (not shown) extending through shell 312 and, in some instances, into core 310.

The presence of bumps (e.g., hemispheric first areas 316) which extend outward relative to second areas 318 of particle 300 can impart enhanced thrombogenicity, aggregation properties, swelling, drug delivery capabilities, and/or potential for use with fibered coil devices. The polymer used can be selected to provide specific characteristics to particles 300. For example, in some embodiments, the first polymer used to form hemispheric first areas 316 can have cationic or anionic properties to selectively dispense therapeutic agents and/or control aggregation characteristics.

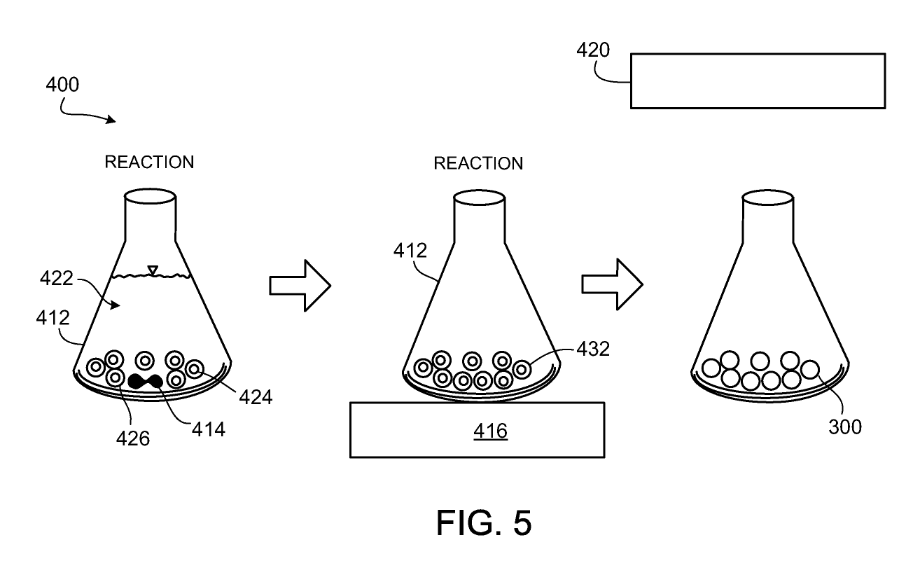

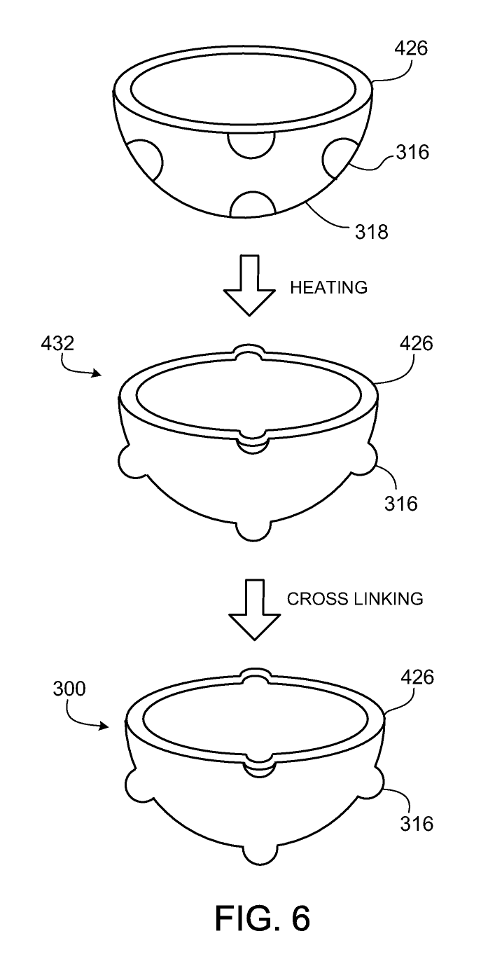

FIGS. 5 and 6, respectively, show a system 400 for producing particles 300 and particles 300 during production. System 400 includes a reaction vessel 412, a mixer 414, a heater 416, and an ultraviolet lamp 420. An aqueous solution is prepared that contains a first amphiphilic diblock copolymer and a second amphiphilic diblock copolymer that share the same water-insoluble block but have different water-soluble blocks. For example, amphiphilic diblock copolymers include, for example, Poly(glyceryl methacrylate)-co-Poly(2-cinnamoylethyl methacrylate), Poly(hydroxyethyl (meth)acrylate)-co-Poly(2-cinnamoylethyl methacrylate), Poly(vinyl pyrrolidone)-co-Poly(2-cinnamoylethyl methacrylate), Poly((meth)acrylamide)-co-Poly(2-cinnamoylethyl methacrylate), Poly(vinyl alcohol)-co-Poly(2-cinnamoylethyl methacrylate). These all contain a UV curable water insoluble block that can be crosslinked to stabilize the particles after formation. In reaction vessel 412, mixer 414 (e.g., a mechanical stirrer or a magnetic stirrer) mixes aqueous solution 422 with an oil phase 424 containing a dissolved polymer. During mixing, a layer 426 including the first amphiphilic diblock copolymer and the second amphiphilic diblock copolymer forms around drops of oil phase 424. The aqueous solution is mixed with the oil phase at rate such that the drops of the oil phase are from 50 to 5,000 microns in size. The size of particles 300 can be increased by decreasing the mixing rate and/or by increasing the concentration of dissolved polymer in the oil phase. Aqueous solution 422 and oil phase 424 are mixed until the first amphiphilic diblock copolymer substantially migrates to first areas 316 of layer 426 and the second amphiphilic diblock copolymer substantially migrates to second areas 318 of layer 426 (e.g., for at least 30 minutes, at least 60 minutes, or at least 120 minutes). Heater 416 increases the temperature in reaction vessel 412 and solidifies intermediate particles 432 by evaporating the oil phase solvent and water out of reaction vessel 412. Ultraviolet lamp 420 irradiates intermediate particles 432 with ultraviolet light and cross-links polymers to form particle 300.

In general, referring to FIGS. 1A, 1B, 4A, and 4B, the maximum dimension of particles 110, 300 is 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the maximum dimension of particles 110, 300 are 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the maximum dimension of particles 110, 300 are less than 100 microns (e.g., less than 50 microns).

In some embodiments, particles 110, 300 can be substantially spherical. In certain embodiments, particles 110, 300 can have a sphericity of 0.8 or more (e.g., 0.85 or more, 0.9 or more, 0.95 or more, 0.97 or more). Particles 110, 300 can be, for example, manually compressed, essentially flattened, while wet to 50 percent or less of its original diameter and then, upon exposure to fluid, regain a sphericity of 0.8 or more (e.g., 0.85 or more, 0.9 or more, 0.95 or more, 0.97 or more). The sphericity of a particle can be determined using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=√(4 A/π); Dp=P/π; A=pixel area; P=pixel perimeter), is a value from zero to one, with one representing a perfect circle.

Examples of polymers include polymers that include vinyl alcohol monomers, vinyl formal monomers and/or vinyl acetate monomers. As referred to herein, a vinyl formal monomer unit has the following structure:

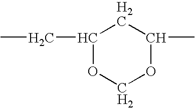

As referred to herein, a vinyl alcohol monomer unit has the following structure:

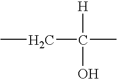

As referred to herein, a vinyl acetate monomer unit has the following structure:

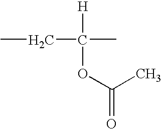

In general, the monomer units can be arranged in a variety of different ways. As an example, in some embodiments, the polymer can include different monomer units that alternate with each other. For example, the polymer can include repeating blocks, each block including a vinyl formal monomer unit, a vinyl alcohol monomer unit, and a vinyl acetate monomer unit. As another example, in certain embodiments, the polymer can include blocks including multiple monomer units of the same type. Generally, however, there should be sufficient PVA present in the polymer to allow the polymer to crystallize.

In some embodiments, the polymer can have the formula that is schematically represented below, in which x, y and z each are integers that are greater than zero. In certain embodiments, x is zero. The individual monomer units that are shown can be directly attached to each other, and/or can include one or more other monomer units (e.g., vinyl formal monomer units, vinyl alcohol monomer units, vinyl acetate monomer units) between them:

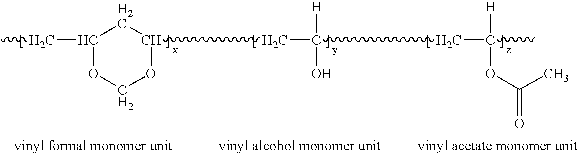

Optionally, formal linkages can occur between PVA molecules giving cross-links.

In some embodiments, the polymer can include at least five percent by weight (e.g., at least 15 percent by weight, at least 25 percent by weight, at least 35 percent by weight) vinyl alcohol monomer units, and/or at most 80 percent by weight (e.g., at most 50 percent by weight, at most 25 percent by weight, at most 10 percent by weight) vinyl alcohol monomer units. The weight percent of a monomer unit in a polymer can be measured using solid-state NMR spectroscopy.

Generally, the polymer will contain little or no vinyl formal monomer units. In some embodiments, the polymer can include at most 10 percent by weight (e.g., at most 5 percent by weight, at most 2 percent by percent by weight) vinyl formal monomer units and/or at least 0.1 percent by weight (e.g., at least 0.5 percent by weight, at least 1 percent by weight) vinyl formal monomer units. As used herein, the weight percent of a monomer unit in a polymer is measured using solid-state NMR spectroscopy as described above.

In some embodiments, the polymer can include at least one percent by weight (e.g., at least two percent by weight, at least five percent by weight, at least 10 percent by weight, at least 15 percent by weight) vinyl acetate monomer units, and/or at most 20 percent by weight (e.g., at most 15 percent by weight, at most 10 percent by weight, at most five percent by weight) vinyl acetate monomer units. As used herein, the weight percent of a monomer unit in a polymer is measured using solid-state NMR spectroscopy as described above.

Other polymers may also be used as a matrix polymer in particles 110, 300. Examples of polymers include polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids (e.g., poly(d-lactic-co-glycolic) acids) and copolymers or mixtures thereof Polymers are described, for example, in Lanphere et al., U.S. Patent Application Publication No. US 2004/0096662 A1, published on May 20, 2004, and entitled “Embolization”; Song et al., U.S. patent application Ser. No. 11/314,056, filed on Dec. 21, 2005, and entitled “Block Copolymer Particles”; and Song et al., U.S. patent application Ser. No. 11/314,557, filed on Dec. 21, 2005, and entitled “Block Copolymer Particles”, all of which are incorporated herein by reference.

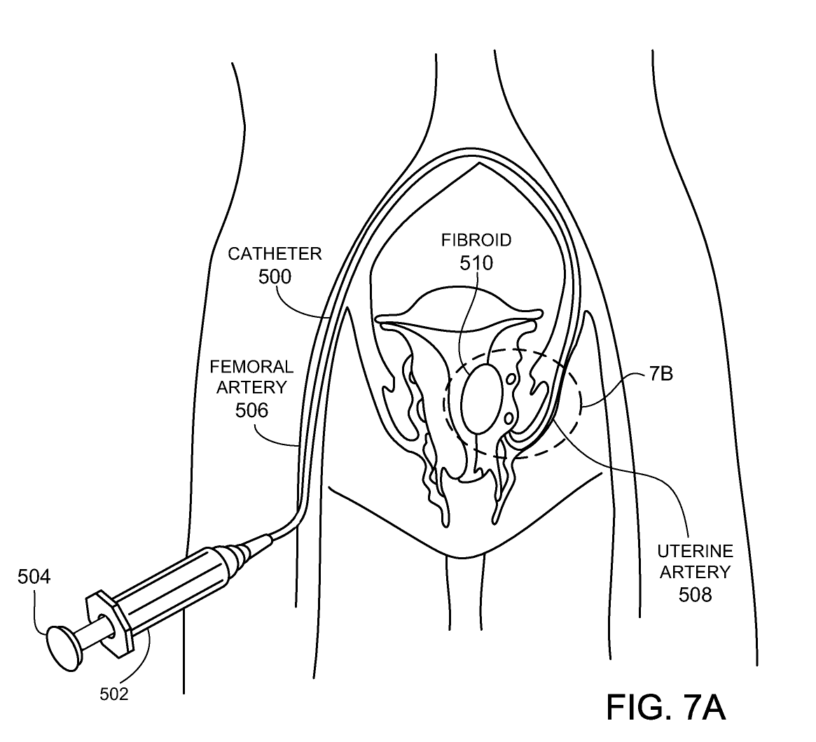

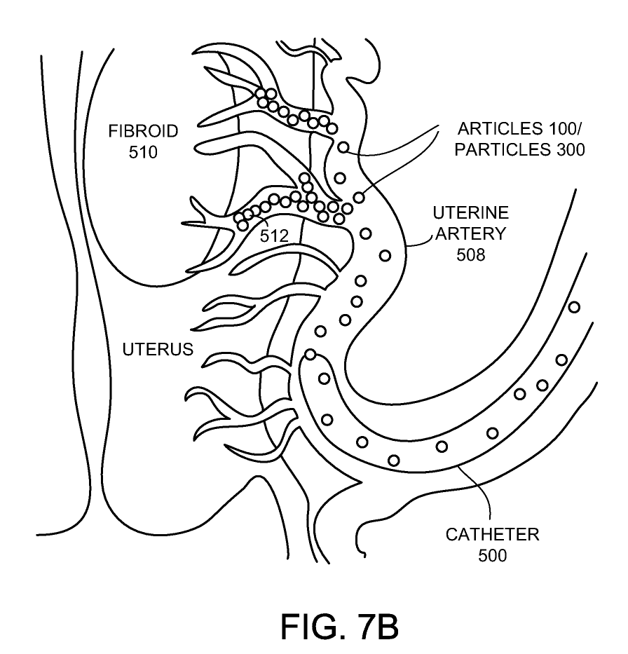

Multiple particles can be combined with a carrier fluid (e.g., a pharmaceutically acceptable carrier, such as a saline solution, a contrast agent, or both) to form a composition, which can then be delivered to a site and used to embolize the site. FIGS. 7A and 7B illustrate the use of a composition including particles to embolize a lumen of a subject. As shown, a composition including articles 100 and/or particles 300 and a carrier fluid is injected into a vessel through an instrument such as a catheter 500. Catheter 500 is connected to a syringe 502 with a plunger 504. Catheter 500 is inserted, for example, into a femoral artery 506 of a subject. Catheter 500 delivers the composition to, for example, occlude a uterine artery 508 leading to a fibroid 510 located in the uterus of a female subject. The composition is initially loaded into syringe 502. Plunger 504 of syringe 502 is then compressed to deliver the composition through catheter 500 into a lumen of uterine artery 508.

FIG. 7B, which is an enlarged view of section 7B of FIG. 7A, shows uterine artery 508, which is subdivided into smaller uterine vessels 512 (e.g., having a diameter of two millimeters or less) that feed fibroid 510. Articles 100 and/or particles 300 in the composition partially or totally fill the lumen of uterine artery 508, either partially or completely occluding the lumen of the uterine artery 508 that feeds uterine fibroid 510.

Compositions including particles such as articles 100 and/or particles 300 can be delivered to various sites in the body, including, for example, sites having cancerous lesions, such as the breast, prostate, lung, thyroid, or ovaries. The compositions can be used in, for example, neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are, for example, abnormal collections of blood vessels (e.g. in the brain) which shunt blood from a high pressure artery to a low pressure vein, resulting in hypoxia and malnutrition of those regions from which the blood is diverted. In some embodiments, a composition containing the particles can be used to prophylactically treat a condition.

The magnitude of a dose of a composition can vary based on the nature, location and severity of the condition to be treated, as well as the route of administration. A physician treating the condition, disease or disorder can determine an effective amount of composition. An effective amount of embolic composition refers to the amount sufficient to result in amelioration of symptoms and/or a prolongation of survival of the subject, or the amount sufficient to prophylactically treat a subject. The compositions can be administered as pharmaceutically acceptable compositions to a subject in any therapeutically acceptable dosage, including those administered to a subject intravenously, subcutaneously, percutaneously, intratrachealy, intramuscularly, intramucosaly, intracutaneously, intra-articularly, orally or parenterally.

A composition can include a mixture of particles (e.g., particles formed of polymers including different weight percents of vinyl alcohol monomer units, particles including different types of therapeutic agents), or can include particles that are all of the same type. In some embodiments, a composition can be prepared with a calibrated concentration of particles for ease of delivery by a physician. A physician can select a composition of a particular concentration based on, for example, the type of procedure to be performed. In certain embodiments, a physician can use a composition with a relatively high concentration of particles during one part of an embolization procedure, and a composition with a relatively low concentration of particles during another part of the embolization procedure.

Suspensions of particles in saline solution can be prepared to remain stable (e.g., to remain suspended in solution and not settle and/or float) over a desired period of time. A suspension of particles can be stable, for example, for from one minute to 20 minutes (e.g. from one minute to 10 minutes, from two minutes to seven minutes, from three minutes to six minutes).

In some embodiments, particles can be suspended in a physiological solution by matching the density of the solution to the density of the particles. In certain embodiments, the particles and/or the physiological solution can have a density of from one gram per cubic centimeter to 1.5 grams per cubic centimeter (e.g., from 1.2 grams per cubic centimeter to 1.4 grams per cubic centimeter, from 1.2 grams per cubic centimeter to 1.3 grams per cubic centimeter).

In certain embodiments, the carrier fluid of a composition can include a surfactant. The surfactant can help the particles to mix evenly in the carrier fluid and/or can decrease the likelihood of the occlusion of a delivery device (e.g., a catheter) by the particles. In certain embodiments, the surfactant can enhance delivery of the composition (e.g., by enhancing the wetting properties of the particles and facilitating the passage of the particles through a delivery device). In some embodiments, the surfactant can decrease the occurrence of air entrapment by the particles in a composition (e.g., by porous particles in a composition). Examples of liquid surfactants include Tween® 80 (available from Sigma-Aldrich) and Cremophor EL® (available from Sigma-Aldrich). An example of a powder surfactant is Pluronic® F127 NF (available from BASF). In certain embodiments, a composition can include from 0.05 percent by weight to one percent by weight (e.g., 0.1 percent by weight, 0.5 percent by weight) of a surfactant. A surfactant can be added to the carrier fluid prior to mixing with the particles and/or can be added to the particles prior to mixing with the carrier fluid.

In some embodiments, among the particles delivered to a subject (e.g., in a composition), the majority (e.g., 50 percent or more, 60 percent or more, 70 percent or more, 80 percent or more, 90 percent or more) of the particles can have a maximum dimension of 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, among the particles delivered to a subject, the majority of the particles can have a maximum dimension of less than 100 microns (e.g., less than 50 microns).

In certain embodiments, the particles delivered to a subject (e.g., in a composition) can have an arithmetic mean maximum dimension of 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the particles delivered to a subject can have an arithmetic mean maximum dimension of less than 100 microns (e.g., less than 50 microns).

Exemplary ranges for the arithmetic mean maximum dimension of particles delivered to a subject include from 100 microns to 500 microns; from 100 microns to 300 microns; from 300 microns to 500 microns; from 500 microns to 700 microns; from 700 microns to 900 microns; from 900 microns to 1,200 microns; and from 1,000 microns to 1,200 microns. In general, the particles delivered to a subject (e.g., in a composition) can have an arithmetic mean maximum dimension in approximately the middle of the range of the diameters of the individual particles, and a variance of 20 percent or less (e.g. 15 percent or less, 10 percent or less).

In some embodiments, the arithmetic mean maximum dimension of the particles delivered to a subject (e.g., in a composition) can vary depending upon the particular condition to be treated. As an example, in certain embodiments in which the particles are used to embolize a liver tumor, the particles delivered to the subject can have an arithmetic mean maximum dimension of 500 microns or less (e.g., from 100 microns to 300 microns; from 300 microns to 500 microns). As another example, in some embodiments in which the particles are used to embolize a uterine fibroid, the particles delivered to the subject can have an arithmetic mean maximum dimension of 1,200 microns or less (e.g., from 500 microns to 700 microns; from 700 microns to 900 microns; from 900 microns to 1,200 microns). As an additional example, in certain embodiments in which the particles are used to treat a neural condition (e.g., a brain tumor) and/or head trauma (e.g., bleeding in the head), the particles delivered to the subject can have an arithmetic mean maximum dimension of less than 100 microns (e.g., less than 50 microns). As a further example, in some embodiments in which the particles are used to treat a lung condition, the particles delivered to the subject can have an arithmetic mean maximum dimension of less than 100 microns (e.g., less than 50 microns). As another example, in certain embodiments in which the particles are used to treat thyroid cancer, the particles can have an arithmetic maximum dimension of 1,200 microns or less (e.g., from 1,000 microns to 1,200 microns). As an additional example, in some embodiments in which the particles are used only for therapeutic agent delivery, the particles can have an arithmetic mean maximum dimension of less than 100 microns (e.g., less than 50 microns, less than 10 microns, less than five microns).

The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the diameters of all of the particles in the group by the number of particles in the group.

Additionally or alternatively to having pores, a particle can have one or more cavities. For example, a particle can be formed so that the polymer surrounds one or more cavities.

A pore has a maximum dimension of at least 0.01 micron (e.g., at least 0.05 micron, at least 0.1 micron, at least 0.5 micron, at least one micron, at least five microns, at least 10 microns, at least 15 microns, at least 20 microns, at least 25 microns, at least 30 microns, at least 35 microns, at least 50 microns, at least 100 microns, at least 150 microns, at least 200 microns, at least 250 microns), and/or at most 300 microns (e.g., at most 250 microns, at most 200 microns, at most 150 microns, at most 100 microns, at most 50 microns, at most 35 microns, at most 30 microns, at most 25 microns, at most 20 microns, at most 15 microns, at most 10 microns, at most five microns, at most one micron, at most 0.5 micron, at most 0.1 micron, at most 0.05 micron).

A cavity has a maximum dimension of at least one micron (e.g., a least five microns, at least 10 microns, at least 25 microns, at least 50 microns, at least 100 microns, at least 250 microns, at least 500 microns, at least 750 microns) and/or at most 1,000 microns (e.g., at most 750 microns, at most 500 microns, at most 250 microns, at most 100 microns, at most 50 microns, at most 25 microns, at most 10 microns, at most five microns). In some embodiments (e.g., when the particle is used to deliver a therapeutic agent within a body lumen, independent of whether embolization is desired), the particle can also include a therapeutic agent (e.g., in one or more pores, in one or more cavities, on the surface of the particle).

Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; proteins; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Examples of radioactive species include yttrium (⁹⁰Y), holmium (¹⁶⁶Ho), phosphorus (³²P), lutetium (¹⁷⁷Lu), actinium (²²⁵Ac), praseodymium, astatine (²¹¹At), rhenium (¹⁸⁶Re), bismuth (²¹²Bi or ²¹³Bi),), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). In some embodiments, yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), and/or rhodium (¹⁰⁵Rh) can be used as therapeutic agents. In certain embodiments, yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), carbon (¹⁴C), and/or tritium (³H) can be used as a radioactive label (e.g., for use in diagnostics). In some embodiments, a radioactive species can be a radioactive molecule that includes antibodies containing one or more radioisotopes, for example, a radiolabeled antibody. Radioisotopes that can be bound to antibodies include, for example, iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁰⁵Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). Examples of antibodies include monoclonal and polyclonal antibodies including RS7, Mov18, MN-14 IgG, CC49, COL-1, mAB A33, NP-4 F(ab′)2 anti-CEA, anti-PSMA, ChL6, m-170, or antibodies to CD20, CD74 or CD52 antigens. Examples of radioisotope/antibody pairs include m-170 MAB with ⁹⁰Y. Examples of commercially available radioisotope/antibody pairs include Zevalin™ (IDEC pharmaceuticals, San Diego, Calif.) and Bexxar™ (Corixa corporation, Seattle, Wash.). Further examples of radioisotope/antibody pairs can be found in J. Nucl. Med. 2003, April: 44(4): 632-40.

Non-limiting examples of therapeutic agents include anti-thrombogenic agents; thrombogenic agents; agents that promote clotting; agents that inhibit clotting; antioxidants; angiogenic and anti-angiogenic agents and factors; anti-proliferative agents (e.g., agents capable of blocking smooth muscle cell proliferation, such as rapamycin); calcium entry blockers (e.g., verapamil, diltiazem, nifedipine); targeting factors (e.g., polysaccharides, carbohydrates); agents that can stick to the vasculature (e.g., charged moieties) (e.g., gelatin, chitosan, collagen, polymers containing bioactive groups like RGD peptides); and survival genes which protect against cell death (e.g., anti-apoptotic Bcl-2 family factors and Akt kinase).

Examples of non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Examples of genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them. Vectors of interest for delivery of genetic therapeutic agents include: plasmids; viral vectors such as adenovirus (AV), adenoassociated virus (AAV) and lentivirus; and non-viral vectors such as lipids, liposomes and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents are disclosed in Kunz et al., U.S. Pat. No. 5,733,925, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

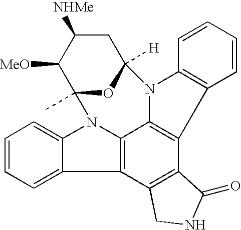

as well as diindoloalkaloids having one of the following general structures:

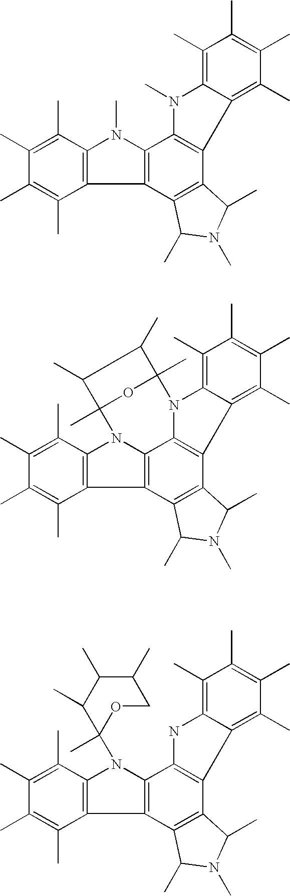

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or—beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including α-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP Iib/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), Fxa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE1 and PGI2; prostacyclins and prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganic ions (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, interleukins, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

In some embodiments, a therapeutic agent can be hydrophilic. An example of a hydrophilic therapeutic agent is doxorubicin hydrochloride. In certain embodiments, a therapeutic agent can be hydrophobic. Examples of hydrophobic therapeutic agents include paclitaxel, cisplatin, tamoxifen, and doxorubicin base. In some embodiments, a therapeutic agent can be lipophilic. Examples of lipophilic therapeutic agents include paclitaxel, other taxane derivative, dexamethasone, other steroid based therapeutics.

Therapeutic agents are described, for example, in DiMatteo et al., U.S. Patent Application Publication No. US 2004/0076582 A1, published on Apr. 22, 2004, and entitled “Agent Delivery Particle”; Schwarz et al., U.S. Pat. No. 6,368,658; Buiser et al., U.S. patent application Ser. No. 11/311,617, filed on Dec. 19, 2005, and entitled “Coils”; and Song, U.S. patent application Ser. No. 11/355,301, filed on Feb. 15, 2006, and entitled “Block Copolymer Particles”, all of which are incorporated herein by reference. In certain embodiments, in addition to or as an alternative to including therapeutic agents, particles 110, 300 can include one or more radiopaque materials, materials that are visible by magnetic resonance imaging (MRI-visible materials), ferromagnetic materials, and/or contrast agents (e.g., ultrasound contrast agents). These materials can, for example, be bonded to the chemical species (monomer(s), oligomers(s), polymer(s)). Radiopaque materials, MRI-visible materials, ferromagnetic materials, and contrast agents are described, for example, in Rioux et al., U.S. Patent Application Publication No. US 2004/0101564 A1, published on May 27, 2004, and entitled “Embolization”, which is incorporated herein by reference.

In certain embodiments, a particle can also include a coating. The coating can, for example, be formed of a polymer (e.g., alginate) that is different from the polymer in main polymer matrix. The coating can, for example, regulate release of therapeutic agent from the particle, and/or provide protection to the interior region of the particle (e.g., during delivery of the particle to a target site). In certain embodiments, the coating can be formed of a bioerodible and/or bioabsorbable material that can erode and/or be absorbed as the particle is delivered to a target site. This can, for example, allow the interior region of the particle to deliver a therapeutic agent to the target site once the particle has reached the target site. A bioerodible material can be, for example, a polysaccharide (e.g., alginate); a polysaccharide derivative; an inorganic, ionic salt; a water soluble polymer (e.g., polyvinyl alcohol, such as polyvinyl alcohol that has not been cross-linked); biodegradable poly DL-lactide-poly ethylene glycol (PELA); a hydrogel (e.g., polyacrylic acid, hyaluronic acid, gelatin, carboxymethyl cellulose); a polyethylene glycol (PEG); chitosan; a polyester (e.g., a polycaprolactone); a poly(ortho ester); a polyanhydride; a poly(lactic-co-glycolic) acid (e.g., a poly(d-lactic-co-glycolic) acid); a poly(lactic acid) (PLA); a poly(glycolic acid) (PGA); or a combination thereof. In some embodiments, the coating can be formed of a swellable material, such as a hydrogel (e.g., polyacrylamide co-acrylic acid). The swellable material can be made to swell by, for example, changes in pH, temperature, and/or salt. In certain embodiments in which the particle is used in an embolization procedure, the coating can swell at a target site, thereby enhancing occlusion of the target site by the particle.

In some embodiments, the coating can be porous. The coating can, for example, be formed of one or more of the above-disclosed polymers.

In certain embodiments, a particle can include a coating that includes one or more therapeutic agents (e.g., a relatively high concentration of one or more therapeutic agents). One or more of the therapeutic agents can also be loaded into the interior region of the particle. Thus, the surface of the particle can release an initial dosage of therapeutic agent, after which the interior region of the particle can provide a burst release of therapeutic agent. The therapeutic agent on the surface of the particle can be the same as or different from the therapeutic agent in the interior region of the particle. The therapeutic agent on the surface of the particle can be applied to the particle by, for example, exposing the particle to a high concentration solution of the therapeutic agent.

In some embodiments, a therapeutic agent coated particle can include another coating over the surface of the therapeutic agent (e.g., a bioerodible polymer which erodes when the particle is administered). The coating can assist in controlling the rate at which therapeutic agent is released from the particle. For example, the coating can be in the form of a porous membrane. The coating can delay an initial burst of therapeutic agent release. In certain embodiments, the coating can be applied by dipping and/or spraying the particle. The bioerodible polymer can be a polysaccharide (e.g., alginate). In some embodiments, the coating can be an inorganic, ionic salt. Other examples of bioerodible coating materials include polysaccharide derivatives, water-soluble polymers (such as polyvinyl alcohol, e.g., that has not been cross-linked), biodegradable poly DL-lactide-poly ethylene glycol (PELA), hydrogels (e.g., polyacrylic acid, hyaluronic acid, gelatin, carboxymethyl cellulose), polyethylene glycols (PEG), chitosan, polyesters (e.g., polycaprolactones), poly(ortho esters), polyanhydrides, poly(lactic acids) (PLA), polyglycolic acids (PGA), poly(lactic-co-glycolic) acids (e.g., poly(d-lactic-co-glycolic) acids), and combinations thereof. The coating can include therapeutic agent or can be substantially free of therapeutic agent. The therapeutic agent in the coating can be the same as or different from an agent on a surface layer of the particle and/or within the particle. A polymer coating (e.g., a bioerodible coating) can be applied to the particle surface in embodiments in which a high concentration of therapeutic agent has not been applied to the particle surface. Coatings are described, for example, in DiMatteo et al., U.S. Patent Application Publication No. US 2004/0076582 A1, published on Apr. 22, 2004, and entitled “Agent Delivery Particle”, which is incorporated herein by reference.

While certain embodiments have been described, other embodiments are possible.

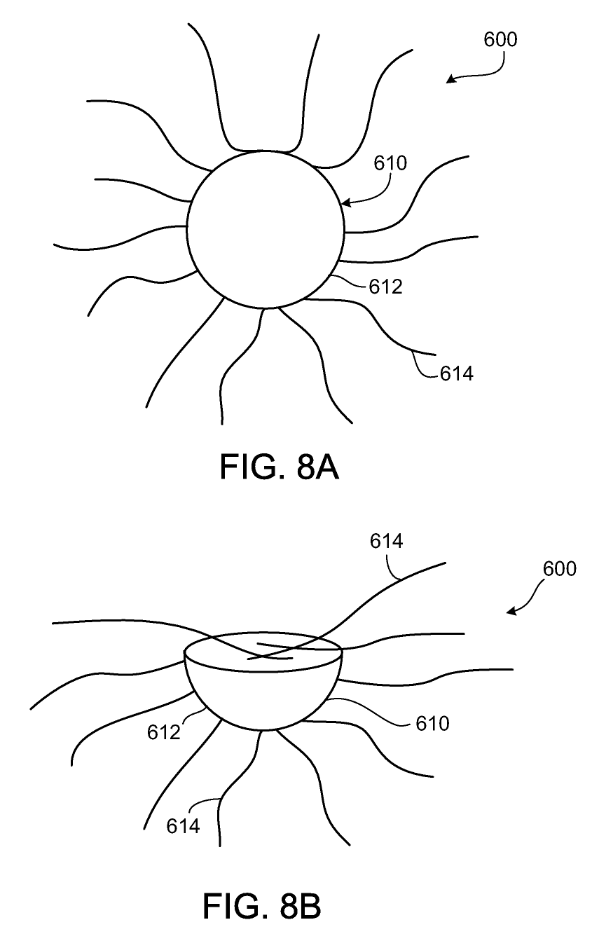

As one example, FIGS. 8A and 8B show an article 600 that can be used, for example, in an embolization procedure. Article 600 includes a particle 610 having an outer surface 612 and fiber members 614 extending outward from outer surface 612 of particle 610. Particle 610 is formed of a polymer, such as polyvinyl alcohol (PVA) which can be cross-linked. Fiber members 614 are disposed partially within particle 610 and can be formed of natural or artificial fibers (e.g., cotton, polyethylene terephthalate, nylon, and collagen). In some embodiments, the article further includes a therapeutic agent (e.g., a therapeutic agent at least partially disposed within pores included in the particle or a therapeutic agent coated on a surface of the particle).

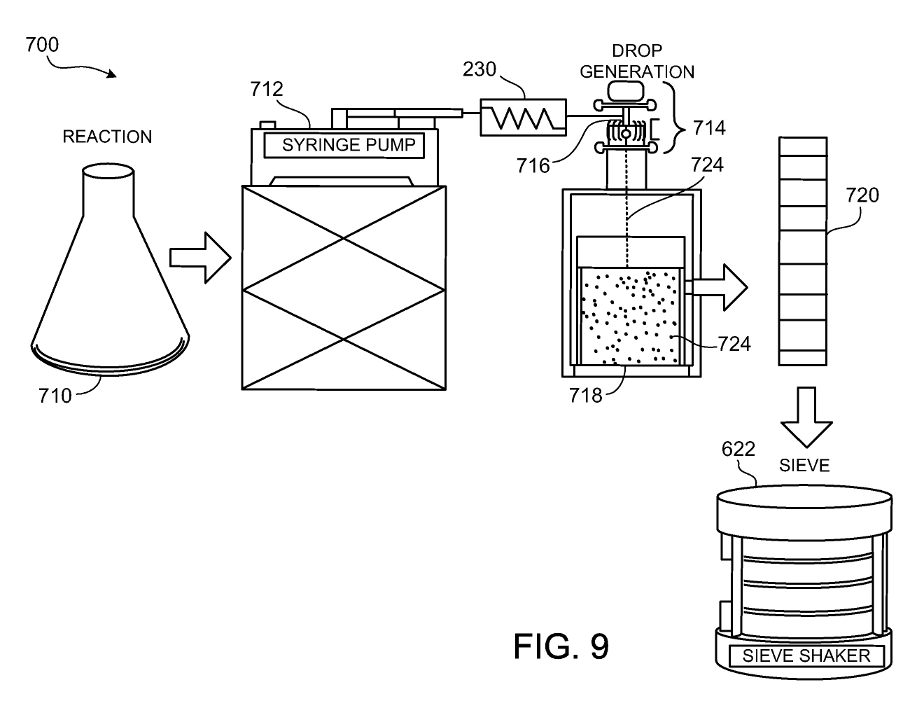

FIG. 9 shows a system 700 for producing articles 600. Fibers are mixed into a polymeric solution including a gelling precursor in reaction vessel 710. Flow controller 712, drop generator 714, and nozzle 716 generate drops 724 which fall into gelling vessel 718 where drops 724 are stabilized by gel formation. During gel formation, the gelling precursor in drops 724 is converted from a solution to a gel form by a gelling agent contained in gelling vessel 718. Particles formed by gel-stabilized drops 724 are then transferred from gelling vessel 718 to gel dissolution chamber 720. In gel dissolution chamber 720, the gelling precursor (which was converted to a gel) in the particles is dissolved leaving articles 600 as particles 610 with fiber members 614 extending outward. After the article formation process has been completed, articles 600 can be filtered in filter 622 to remove debris.

As another example, in some embodiments, particles can be used for tissue bulking. As an example, the particles can be placed (e.g., injected) into tissue adjacent to a body passageway. The particles can narrow the passageway, thereby providing bulk and allowing the tissue to constrict the passageway more easily. The particles can be placed in the tissue according to a number of different methods, for example, percutaneously, laparoscopically, and/or through a catheter. In certain embodiments, a cavity can be formed in the tissue, and the particles can be placed in the cavity. Particle tissue bulking can be used to treat, for example, intrinsic sphincteric deficiency (ISD), vesicoureteral reflux, gastroesophageal reflux disease (GERD), and/or vocal cord paralysis (e.g., to restore glottic competence in cases of paralytic dysphonia). In some embodiments, particle tissue bulking can be used to treat urinary incontinence and/or fecal incontinence. The particles can be used as a graft material or a filler to fill and/or to smooth out soft tissue defects, such as for reconstructive or cosmetic applications (e.g., surgery). Examples of soft tissue defect applications include cleft lips, scars (e.g., depressed scars from chicken pox or acne scars), indentations resulting from liposuction, wrinkles (e.g., glabella frown wrinkles), and soft tissue augmentation of thin lips. Tissue bulking is described, for example, in Bourne et al., U.S. Patent Application Publication No. US 2003/0233150 A1, published on Dec. 18, 2003, and entitled “Tissue Treatment”, which is incorporated herein by reference.

As an additional example, in certain embodiments, particles can be used to treat trauma and/or to fill wounds. In some embodiments, the particles can include one or more bactericidal agents and/or bacteriostatic agents.

As a further example, while compositions including particles suspended in at least one carrier fluid have been described, in certain embodiments, particles may not be suspended in any carrier fluid. For example, particles alone can be contained within a syringe, and can be injected from the syringe into tissue during a tissue ablation procedure and/or a tissue bulking procedure.

As an additional example, in some embodiments, particles having different shapes, sizes, physical properties, and/or chemical properties can be used together in a procedure (e.g., an embolization procedure). The different particles can be delivered into the body of a subject in a predetermined sequence or simultaneously. In certain embodiments, mixtures of different particles can be delivered using a multi-lumen catheter and/or syringe. In some embodiments, particles having different shapes and/or sizes can be capable of interacting synergistically (e.g., by engaging or interlocking) to form a well-packed occlusion, thereby enhancing embolization. Particles with different shapes, sizes, physical properties, and/or chemical properties, and methods of embolization using such particles are described, for example, in Bell et al., U.S. Patent Application Publication No. US 2004/0091543 A1, published on May 13, 2004, and entitled “Embolic Compositions”, and in DiCarlo et al., U.S. Patent Application Publication No. US 2005/0095428 A1, published on May 5, 2005, and entitled “Embolic Compositions”, both of which are incorporated herein by reference.

As a further example, in some embodiments in which a particle including a polymer is used for embolization, the particle can also include (e.g., encapsulate) one or more embolic agents, such as a sclerosing agent (e.g., ethanol), a liquid embolic agent (e.g., n-butyl-cyanoacrylate), and/or a fibrin agent. The other embolic agent(s) can enhance the restriction of blood flow at a target site.

As another example, in some embodiments, a treatment site can be occluded by using particles in conjunction with other occlusive devices. For example, particles can be used in conjunction with coils. Coils are described, for example, in Elliott et al., U.S. patent application Ser. No. 11/000,741, filed on Dec. 1, 2004, and entitled “Embolic Coils”, and in Buiser et al., U.S. patent application Ser. No. 11/311,617, filed on Dec. 19, 2005, and entitled “Coils”, both of which are incorporated herein by reference. In certain embodiments, particles can be used in conjunction with one or more gels. Gels are described, for example, in Richard et al., U.S. Patent Application Publication No. US 2006/0045900 A1, published on Mar. 2, 2006, and entitled “Embolization”, which is incorporated herein by reference. Additional examples of materials that can be used in conjunction with particles to treat a target site in a body of a subject include gel foams, glues, oils, and alcohol.

As a further example, while particles including a polymer have been described, in some embodiments, other types of medical devices and/or therapeutic agent delivery devices can include such a polymer. For example, in some embodiments, a coil can include a polymer as described above. In certain embodiments, the coil can be formed by flowing a stream of the polymer into an aqueous solution, and stopping the flow of the polymer stream once a coil of the desired length has been formed. Coils are described, for example, in Elliott et al., U.S. patent application Ser. No. 11/000,741, filed on Dec. 1, 2004, and entitled “Embolic Coils”, and in Buiser et al., U.S. patent application Ser. No. 11/311,617, filed on Dec. 19, 2005, and entitled “Coils”, both of which are incorporated herein by reference. In certain embodiments, sponges (e.g., for use as a hemostatic agent and/or in reducing trauma) can include a polymer as described above. In some embodiments, coils and/or sponges can be used as bulking agents and/or tissue support agents in reconstructive surgeries (e.g., to treat trauma and/or congenital defects).

Other embodiments are in the claims.

1. An article comprising:
a particle comprising a first polymer, the particle having an outer surface, and the particle having a maximum dimension from 50 to 5,000 microns; and
a first polymer chain comprising a second polymer extending radially outward from the outer surface of the particle.

2-4. (canceled)

5. The article of claim 1, wherein the second polymer has a volume phase transition temperature from 35 to 40 degrees Celsius.

6. The article of claim 1, further comprising a second polymer chain comprising a third polymer.

7. The article of claim 6, wherein the second polymer is soluble in water at 20 degrees Celsius and the third polymer is insoluble in water at 20 degrees Celsius.

8. The article of claim 1, wherein the first polymer comprises vinyl alcohol monomers.

9. (canceled)

10. The article of claim 1, wherein the first polymer further comprises vinyl acetate monomer units.

11. (canceled)

12. The article of claim 1, wherein the article further comprises a therapeutic agent.

13. The article of claim 12, wherein the therapeutic agent is at least partially disposed within pores included in the particle.

14. The article of claim 12, wherein the therapeutic agent is coated on a surface of the particle.

15. The article of claim 12, wherein the therapeutic agent is at least partially disposed within pores in the polymer chain.

16. (canceled)

17. The article of claim 1, wherein the first polymer is different than the second polymer.

18-21. (canceled)

22. An article comprising:
a particle comprising a polymer, the particle having an outer surface, and the particle having a maximum dimension from 50 to 5,000 microns; and
a fiber member extending radially outward from the outer surface of the particle.

23. The article of claim 22, wherein the fiber member is disposed partially within the particle.

24. The article of claim 22, wherein the fiber member comprises a material selected from the group consisting of cotton, polyethylene terephthalate, nylon, and collagen.

25-28. (canceled)

29. The article of claim 22, wherein the article further comprises a therapeutic agent.

30-32. (canceled)

33. A particle comprising:
a core having an outer surface, and a core size from 50 to 5,000 microns; and
a shell formed on the outer surface of the core, a first area of the shell comprising a first polymer and a second area of the shell comprising a second polymer different than the first polymer; the first area extending radially outward an average first distance from a center of the core and the second area extending radially outward an average second distance from the center of the core, the average second distance being greater than the average second distance.

34. The particle of claim 33, wherein the particle includes pores extending through the shell.

35. The particle of claim 34, wherein the pores extend into the core.

36. The particle of claim 33, wherein the first distance is at least 10 percent greater than the second distance.

37. The particle of claim 36, wherein the first distance is less than 50 percent greater than the second distance.

38-62. (canceled)
